(19) United States
(12) Patent Application Publication
(10) Pub. No.: US 2022/0202495 A1
(43) Pub. Date: Jun. 30, 2022

(54) PRECISE TUNNEL LOCATION PLACEMENT AND GUIDANCE FOR A ROBOTIC DRILL

(71) Applicant: THINK SURGICAL, INC., Fremont, CA (US)

(72) Inventor: Timothy J. Pack, Fremont, CA (US)

(73) Assignee: THINK SURGICAL, INC., Fremont, CA (US)

(21) Appl. No.: 17/603,455

(22) PCT Filed: May 13, 2020
(86) PCT No.: PCT/US2020/032561

§ 371 Date: Oct. 13, 2021

Related U.S. Application Data
(60) Provisional application No. 62/847,036, filed on May 13, 2019.

(51) Int. Cl. A61B 34/10; A61B 34/30; A61B 34/20; A61B 17/32

(52) U.S. Cl. CPC A61B 34/10; A61B 34/30; A61B 34/20; A61B 2017/320056; A61B 2034/107; A61B 2034/2055; A61B 17/32

(57) ABSTRACT

A system and method are described herein for designating a precise location for a bone tunnel to receive a ligament or tendon graft and guiding a robotic drill for creating the bone tunnel. The system includes a tracking system, a digitizer, a computing system, and a robotic drill. The digitizer designates a start point for the tunnel entry site and an end point where the tunnel terminates. The start point and end point are recorded in the computing system and a vector is calculated between the start point and the end point. The vector is supplied to a robotic drill to drill the tunnel in the bone. A ligament or tendon graft may be placed in the tunnel and secured to the bone to complete the procedure.

### RELATED APPLICATIONS

This application claims priority benefit of U.S. Provisional Application Ser. No. 62/847,036, filed 13 May 2019, the contents of which are hereby incorporated by reference.

### TECHNICAL FIELD

The present invention generally relates to computer assisted surgery, and more specifically to systems and methods for precise tunnel location placement and guiding of a robotic drill for creating a tunnel for an intended ligament graft during a surgical procedure.

### BACKGROUND

Rupture of the anterior cruciate ligament (ACL) is one of the most frequent injuries to the knee joint. ACL reconstruction is a major orthopedic procedure most often performed to repair the knee joint. Early stabilization of the knee joint by ACL reconstruction also decreases the risk of injury to other important structures.

The goal of anterior cruciate ligament (ACL) reconstruction procedures, as well as other ligament and tendon repairs to joints including the elbow, is to replace a ruptured ligament or tendon with a graft that provides similar mechanical stability of the native anatomy while preserving the range of motion of the knee or joint. However, the native cruciate ligature of the knee is highly complex, and presents several challenges for successful reconstruction procedures.

During ACL reconstruction procedures a graft is placed into roughly the same location that the native ACL occupied prior to rupture. To achieve this colocation with a graft, holes are drilled in the femur and tibia in the approximate footprint of the native ACL. A graft is placed in these tunnels, and fixated by some means on both ends. The intent of the graft is to restore stability to the injured knee, while maintaining range of motion.

However, the biggest challenge in ACL reconstruction is typically the exact placement of drilled bone tunnels. When poorly placed, bone tunnels significantly affect the outcome of surgery. Outcomes affected by poor tunnel placement include restricted range of motion, knee joint instability, reaction of the synovium in the knee, and knee joint pain. Furthermore, impingement of the graft and/or improper graft tension may result in potential graft failure with lesion development. A study entitled “Tunnel position and graft orientation in failed anterior cruciate ligament reconstruction: a clinical and imaging analysis” (Ali Hosseini et al., International Orthopaedics 2012 April; 36(4): 845-852) confirmed that technical errors in positioning of graft tunnels is the most common problem in ACL reconstruction. The study quantitatively evaluated femoral and tibial tunnel positions and intra-articular graft orientation of primary ACL reconstruction in patients who had undergone revision ACL reconstruction. The study found that nonanatomically positioned tunnel and graft orientation was a primary cause of graft failure. It was further determined that the sagittal elevation angle for failed ACL reconstruction graft (69.6°±13.4°) was significantly greater (p<0.05) than that of the native anteromedial (AM) and posterolateral (PL) bundles of the ACL (AM 56.2°±6.1°, PL 55.5°±8.1°). In the transverse plane, the deviation angle of the failed graft (37.3°±21.0°) was significantly greater than native ACL bundles.

Precisely placed bone tunnels are difficult to achieve through current surgical methods. While ACL reconstruction is predominately performed arthroscopically, arthroscopy does not allow the surgeon to gain a complete 3D view of important anatomical structures, particularly in the anteroposterior direction. Large incisions are often required to provide surgeons adequate access to landmarks and/or drill angles. Further, as ACL reconstructions require a high learning curve to master, attainable only from high volumes and extensive experience, ACL reconstructions are most often performed by under experienced orthopedic surgeons. It is estimated that up to 20% of ACL grafts fail due to impingement, improper graft tension, or poor tunnel placement.

Furthermore, manual drilling of the tunnels for ligament and tendon placement and fixation is error prone due to changes in bone density and hardness as a drill bit progresses through a bone. In addition the uneven and slippery surfaces of the cartilage at bone joints make manual drilling difficult.

Various techniques have been developed to help a surgeon correctly plan and create bone tunnels for implantation and attachment of ligaments to the bones of a joint. One system and method to optimize ligament reconstruction surgical outcomes is achieved by enabling bone tunnels to be precisely and optimally placed through the use of pre-operative planning systems coupled with precision control bone evacuation machines, such as robotic drills is described in U.S. Pat. No. 10,034,675 assigned to the assignee of the present application, which is incorporated herein in its entirety by reference. In order for the robotic drill to accurately prepare the tunnel, the bone needs to be registered to the surgical plan. Registration maps the surgical plan to the spatial position and orientation (POSE) of the bone in a coordinate system of the surgical system. However, several of these registration techniques require a large exposure of the bone to permit a user to collect a plurality of points on the bone to facilitate the registration. This may be undesirable considering ligament reconstruction surgery is conventionally performed in a minimally invasive manner.

While there have been advancements in ligament replacement and reconstructive surgeries, there exists a need for a system and method for robotic drilling of bone tunnels for intended ligament or tendon grafts that does not require preoperative scanning.

### SUMMARY

A method for precise tunnel location placement and guiding of a robotic drill for creating a tunnel in a bone of a patient is described herein. A digitizer designates a start point for a first tunnel entry site for a tunnel in the first bone. The digitizer designates an end point where the first tunnel terminates on the first bone. The start and end points are supplied to the robotic drill as a first vector to drill the first tunnel with a length and a direction.

A computer-assisted surgical system for designating a precise location for a tunnel and guiding a robotic drill for creating a tunnel in a bone is described herein. The system includes a tracking system, a tracked digitizer, a computing system, and a robotic drill. The tracking system tracks the position of the digitizer to determine the location of the start point and end point designated by the digitizer. The computing system has one or more computers with software, where the one or more computers record the location of the designated start point and the designated end point, and calculates a first vector between the start point and the end point. The computing system further supplies the vector to the robotic drill for drilling a tunnel for an intended ligament graft or tendon graft

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 illustrates a knee joint with markers attached to a tibia bone T and a femur bone F, and a digitizer for establishing entry and exit points for tunnels to be drilled in the bones in accordance with embodiments of the invention;

FIG. 2 illustrates a reconstructive ligament/tendon implant graft inserted in an intended position of a native anterior cruciate ligament (ACL) illustrating the drilled tunnels in accordance with embodiments of the invention;

FIG. 3 depicts a method for making bone tunnels during a robotic assisted surgical procedure in accordance with embodiments of the invention;

FIG. 4 depicts a surgical system in the context of an operating room (OR) with a surgical robot for implementing the method of FIG. 3 in accordance with embodiments of the invention.

### DETAILED DESCRIPTION

The present invention has utility as a system and method for guiding a robotic drill through a precise location without the need for a preoperative scan when forming bone tunnels for fixation of ligaments and tendons. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to anterior cruciate ligament (ACL) reconstruction procedures, the systems and methods may be applied to other computer-assisted surgical procedures involving other ligatures and tendons involved with joints in the body illustratively including the hip, ankle, elbow, wrist, as well as revision of initial repair or replacement of any joints.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “digitizer” refers to a device capable of measuring, collecting, designating, or recording the position of physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described in, for example, U.S. Pat. No. 7,043,961; a digitizer probe as described in U.S. Pat. No. 8,615,286; or an end-effector of a robotic device.

As used herein, the term “digitizing” refers to the collecting, measuring, designating, and/or recording of physical points in space with a digitizer.

Also described herein are “robotic surgical systems”. A robotic surgical system refers to a system (or device) requiring computer control of an end-effector to aid in a surgical procedure. Examples of a robotic surgical systems include active or haptic 1 to N degree(s) of freedom (DOF) hand-held surgical devices and systems, autonomous serial-chain manipulator systems, haptic serial chain manipulator systems, parallel robotic systems, master-slave robotic systems, etc., as described in, for example, U.S. Pat. Nos. 5,086,401; 7,206,626; 8,876,830; 8,961,536; and 9,707,043; and U.S. Pat. App. Pub. US20180344409A1, which patents, patent publications and patent applications are hereby incorporated herein by reference. The surgical system may provide autonomous/automatic, semi-autonomous/automatic, power, or haptic control, and any combinations thereof. In addition, a user may manually maneuver a tool attached to the surgical system while the system provides at least one of power control, active control, guidance control, or haptic control to the tool. In particular embodiments, the robotic surgical device is automatically or semi-automatically controlled such as the robotic surgical system described in detail below.

As used herein, the term “real-time” refers to the processing of input data within milliseconds such that calculated values are available within 10 seconds of computational initiation.

As used herein, the term “tracking reference device” refers to a reference device capable of being tracking by a tracking system. Examples of a tracking reference device include a fiducial marker, a tracking array, an electromagnetic transmitter, a distal end of a mechanical tracking system, or any other device attached, attachable, or integrated on a bone or an object for the proposes of permitting a tracking system to track the bone or the object. In particular, a “tracking array” refers to three or more fiducial markers (e.g., light emitting diodes (LEDs), retroreflective spheres) arranged on a rigid body, or arranged directly on/with an object to be tracked.

Also used herein is the term “optical communication” which refers to wireless data transfer via infrared or visible light as described in U.S. Pat. App. Publication 20170245945 assigned to the assignee of the present application and incorporated by reference herein in its entirety.

Embodiments of the invention establish a tool path for an automated drill that doesn't require all of the steps of traditional registration when drilling ligament or tendon fixation points or tunnels in a patient's bone. In embodiments of the inventive method tracking reference devices are rigidly attached to bones, and the tracking reference devices are tracked with a tracking system to provide a reference frame. A digitizer is then used to collect two points on the bone that establish a start point and an end point for a tunnel to be created with a robotic drill. For example, in an ACL surgery, where separate tibial and femoral tunnels are required, a tracking reference device is fixated rigidly to each of the operative bones. The tracking reference devices are tracked with a tracking system (e.g., an optical tracking system, electromagnetic tracking system). A digitizer is used to designate an entry point in the center of the desired tunnel entry site of the tibia, where the location of the designated entry point is recorded in a computing system. The coordinates of the designated entry point may be recorded relative to the coordinate system of the tracking reference device. The digitizer is also used to designate an exit point where the tunnel terminates. The location of the designated exit point is recorded in a computing system, where the coordinates of the exit point is recorded relative to the coordinate system of the tracking reference device. Once the two points are recorded, the computing system calculates a vector between the points. This vector establishes tunnel direction and length for the tunnel to be drilled in the tibia. The tracking reference device provides a reference frame to track the vector during drilling. In a similar manner, two points (entry, exit) are established on the femur in order to guide the robotic drill when forming a tunnel in the femur.

Embodiments of the invention allow for precise tunnel placement without the need of a pre-operative scan or registration. In the case of an ACL reconstruction, real time tracking information can be used to determine if good locations for tunnels have been chosen.

In addition, the distance between the two tunnel entry points into the knee joint may be monitored throughout a full range of motion to ensure that it is isometric throughout the full range of motion. This provides the surgeon with confirmation of good tunnel placement prior to drilling.

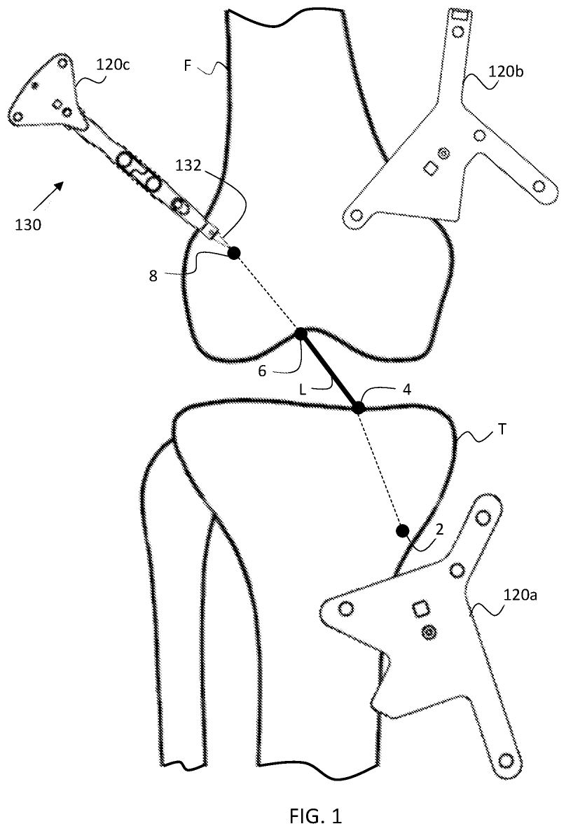

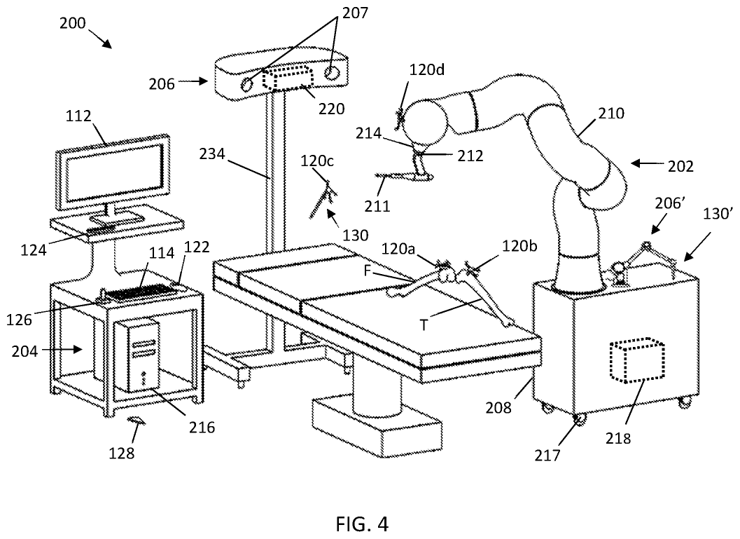

Referring now to the figures, FIG. 1 illustrates a tibia bone T and a femur bone F each with a rigidly affixed tracking array (120a, 120b), respectively. A tracked digitizer probe 130 having a probe tip 132 is used to establish a tibial tunnel entry point 2 and a tibial tunnel exit point 4, as well as a femoral entry point 6 and a femoral exit point 8. The start and end points are used to calculate a vector for a robotic drill (shown as 211 in FIG. 4) to drill tunnels with a length and direction in the tibia and femoral bones. Line L between tibial tunnel exit point 4 and femoral entry point 6 represents the position and length of the native ligament or tendon (which may or may not be removed prior to designating the entry and exit points).

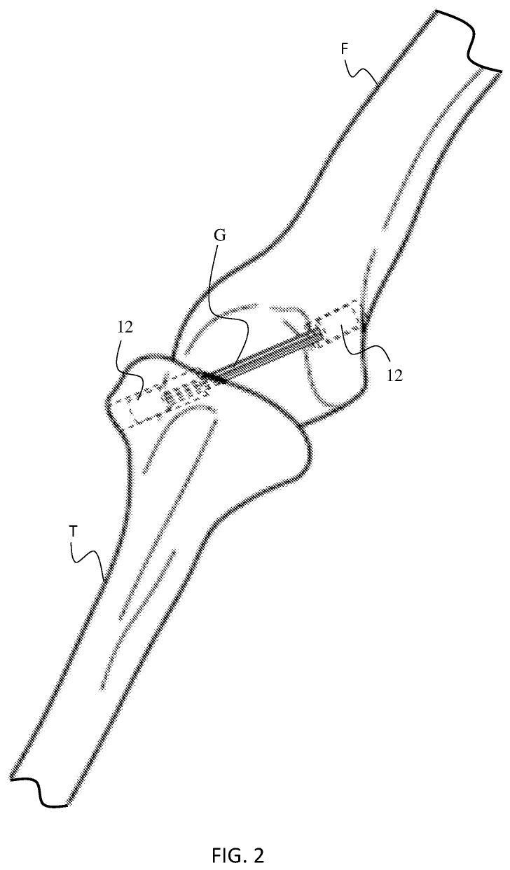

FIG. 2 illustrates a postsurgical view of a ligament/tendon graft G inserted in the tunnels. The final position of the ligament/tendon graft G may coincide with the position of the native anterior cruciate ligament (ACL) or a position desired by the user. Fixation securements 12 are attached to the ends of the graft G in the distal femur F and proximal tibia T, respectively, to secure the graft G in place.

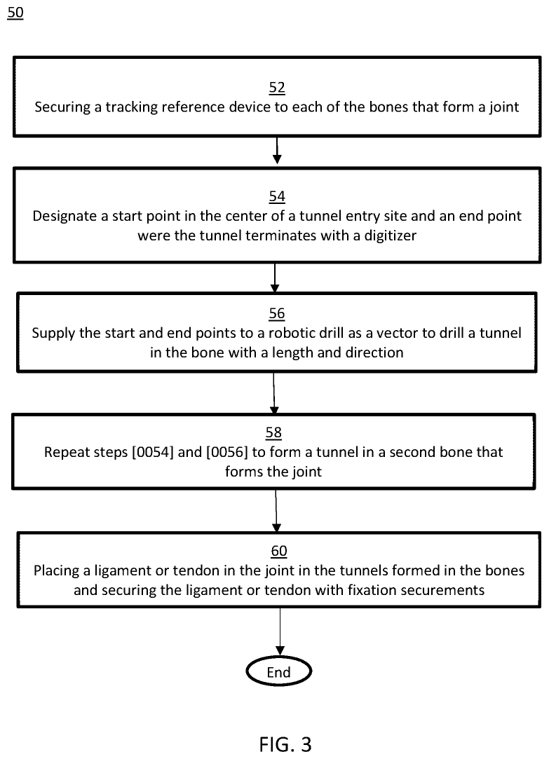

FIG. 3 depicts an embodiment of a method 50 for guiding a robotic drill through a precise location without the need for a preoperative scan when forming bone tunnels for fixation of ligaments and tendons in the context of ACL replacement surgery. A first tracking reference device is fixedly secured to a first bone, and a second tracking reference device is fixedly secured to a second bone, where the first bone and second bone form a joint (Block 52). A digitizer is used to designate a start point in the center of the desired tunnel entry site of the first bone, and an end point where the tunnel terminates on the first bone (Block 54). The locations of the designated start point and end point are recorded in a computing system. The start and end points are supplied to the robotic drill as a vector to drill tunnels with a length and direction (Block 56). The steps [0054]-[0056] are repeated to form a tunnel in the second bone (Block 62). The ligament/tendon is placed in the joint in the tunnels formed in the first and second bones and secured with fixation securements (Block 64).

FIG. 4 depicts a surgical system 200 for implementing the embodiments of the method of FIG. 3. The surgical system 200 includes a surgical robot 202, a computer system 204, and a tracking system 206. The surgical robot 202 may include a movable base 208, a manipulator arm 210 connected to the base 208, an end-effector 211 located at a distal end 212 of the manipulator arm 210, and a force sensor 214 positioned proximal to the end-effector 211 for sensing forces experienced on the end-effector 211. In embodiments of the invention, the end-effector 211 is a drill for forming tunnels in bone for fixation of ligaments and tendons. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 211 in various degrees of freedom. The joints are illustratively prismatic, revolute, spherical, or a combination thereof. The surgical robot 202 may further include a tracking reference device 120d to permit the tracking system 206 to track the position and orientation of the end-effector 211.

The computing system 204 generally includes an optional planning computer 216; a device computer 218; and tracking computer 220; and peripheral devices. The planning computer 216, device computer 218, and tracking computer 220 may be separate entities, one-in-the-same, or combinations thereof depending on the surgical system. Further, in some embodiments, any combination of the planning computer 216, the device computer 218, and/or tracking computer 220 are connected via a wired or wireless communication. The peripheral devices allow a user to interface with the surgical system components and may include: one or more user-interfaces, such as a display or monitor 112 for the graphical user interface (GUI); and user-input mechanisms, such as a keyboard 114, mouse 122, pendant 124, joystick 126, foot pedal 128, or the monitor 112 that in some inventive embodiments has touchscreen capabilities.

The planning computer 216 is optional in that the methods described herein (e.g., the method of FIG. 3) may be performed without pre-operative or intra-operative planning. However, in some instances, a surgeon may choose to review pre-operative images prior to the procedure to gauge or plan the location for the entry and exit points on the one or more bones. Therefore, the optional planning computer 216 may contain hardware (e.g., processors, controllers, and/or memory), software, data and utilities that are in some inventive embodiments dedicated to the review of any pre-operative or intra-operative images and to plan the location for the entry points and exit points. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include image bone data, patient data, ligature implant and tunnel position data, trajectory parameters, and/or operational data. The surgical plan data generated from the planning computer 216 may be displayed during the surgical procedure to assist the surgeon. If the planning computer 216 is located outside the OR, the surgical plan data may be transferred to the device computer 218, tracking computer 220, or other computer in communication with an OR display by way of a non-transient data storage medium (e.g., a compact disc (CD), a portable universal serial bus (USB) drive).

The device computer 218 in some inventive embodiments is housed in the moveable base 208 and contains hardware, software, data and utilities that are preferably dedicated to the operation of the surgical robotic device 202. This may include end-effector control, robotic manipulator control, the processing of kinematic and inverse kinematic data, the execution of calibration routines, the execution of operational data (e.g., trajectory parameters, guidance control), coordinate transformation processing, providing workflow instructions to a user, and utilizing position and orientation (POSE) data from the tracking system 206. In particular embodiments, the device computer 218 records the entry point and exit point designated by the digitizer, and calculates the vector between the entry point and exit point.

The surgical system 200 further includes a digitizer 130. The digitizer 130 may be a mechanically tracked digitizer probe 130′ or an optically tracked digitizer probe 130. The digitizer 130 is used to designate a start point in the center of the desired tunnel site entry site and an end point where the tunnel terminates on the one or more bones. The optically tracked digitizer probe 130 includes a tracking reference device 120c to permit an optical tracking system 206 to track the position and orientation of the probe 203 and the probe tip. The mechanically tracked digitizer probe 130′ is tracked by a mechanical tracking arm 206′ having a plurality of joints, links, and encoders to track the position of the digitizer probe 130′. If a mechanically tracked digitizer probe 130′ is used, then the bones may be fixed into position relative to the surgical robot 202 using fixation hardware as described in U.S. Pat. No. 5,086,401.

In particular embodiments, the tracking system 206 is an optical tracking system that includes two or more optical receivers 207 to detect the position of fiducial markers (e.g., retroreflective spheres, active light emitting diodes (LEDs)) uniquely arranged on rigid bodies. The fiducial markers arranged on a rigid body are collectively referred to as a tracking array (120a, 120b, 120c, 120d), where each tracking array has a unique arrangement of fiducial markers, or a unique transmitting wavelength/frequency if the markers are active LEDs. An example of an optical tracking system is described in U.S. Pat. No. 6,061,644. The tracking system 206 may be built into a surgical light, located on a boom, a stand 234, or built into the walls or ceilings of the OR. The tracking system computer 220 may include tracking hardware, software, data, and utilities to determine the POSE of objects (e.g., bones B, surgical device 202) in a local or global coordinate frame. The POSE of the objects is collectively referred to herein as POSE data or tracking, where this POSE data may be communicated to the device computer 218 through a wired or wireless connection. The wireless communication may be accomplished via optical communication. Alternatively, the device computer 218 may determine the POSE data using the position of the fiducial markers detected from the optical receivers 207 directly.

The POSE data is determined using the position data detected from the optical receivers 207 and operations/processes such as image processing, image filtering, triangulation algorithms, geometric relationship processing, registration algorithms, calibration algorithms, and coordinate transformation processing.

The POSE data is used by the computing system 204 during the procedure to update the POSE and/or coordinate transforms of the vector (or entry and exit points) and the surgical robot 202 as the manipulator arm 210 and/or bone(s) (F, T) move during the procedure, such that the surgical robot 202 can accurately drill the tunnels in the designated locations.

In particular embodiments, the tracking system computer 220 records the location of the entry point and exit point designated by the digitizer probe 130 and calculates the vector therebetween. The optical tracking system 206 may then send informational data, tracking data, and/or operational data to the device computer 218 to control or assist in the control of the end-effector 211 in creating the tunnels in the designated locations.

In particular inventive embodiments, the surgical robot 202 utilizes semi-active control to create the tunnels in the designated location. After the vector is calculated between the designated entry point and exit point, the surgical robot 202 automatically aligns the longitudinal axis of the end-effector 211 coincident with the calculated vector. The end-effector 211 may be positioned proximal to the bones, avoiding any soft tissue, prior to turning-on the end-effector 211 (e.g., prior to turning-on the drill). A user may the hand-guide the end-effector 211 into the bone, where the surgical robot 202 maintains the end-effector's movement along the calculated vector. In other words, the surgical robot 202 prohibits any movement of the end-effector 211 deviating from the calculated vector. If the bone moves while drilling the tunnel, the tracking system 206 can update the position of the vector in real-time based on the movements of the tracking reference devices. Once the end-effector 211 reaches the exit point, the surgical robot 202 stops the end-effector 211 such that the user can no longer guide the end-effector 202 past the exit point. After the tunnel is created, the user may hand-guide the end-effector 211 from the bone while the surgical robot 202 maintains the end-effector 211 along the vector.

The surgical robot 202 may alternatively utilize active control to create the tunnels in the designated location. After the vector is calculated between the designated entry point and exit point, the surgical robot 202 automatically aligns the longitudinal axis of the end-effector 211 coincident with the calculated vector. The end-effector 211 may be positioned proximal to the bones, avoiding any soft tissue, prior to turning-on the end-effector 211 (e.g., prior to turning-on the drill). The surgical robot 202 then automatically controls the end-effector along the calculated vector to create the tunnel. The surgical robot 202 maintains the end-effector 211 along the calculated vector while drilling, stops at the exit point, and retracts once completed. If the bone moves while drilling the tunnel, the tracking system 206 can update the position of the vector in real-time based on the movements of the tracking reference devices.

The surgical robot 202 may alternatively utilize haptic control to create the tunnels in the designated location. After the vector is calculated between the designated entry point and exit point, a user wielding the end-effector 211 is haptically constrained to the calculated vector. If the user deviates from the vector, the surgical robot produces a counter force to constrain the user to the calculated vector. A counter force is likewise produced when the end-effector 211 reaches the exit point. If the bone moves while drilling the tunnel, the tracking system 206 can update the position of the vector in real-time based on the movements of the tracking reference devices.

Other robotic surgical systems may also be used to create the tunnels in the designated location. The robotic systems may provide power control (e.g., cut power to the drill when the exit point is reached), active control, semi-active control, or haptic control.

In a specific embodiment, the robotic surgical system 200 utilizes a mechanically tracked digitizer probe 130′ and fixation hardware for fixing the bones relative to the surgical robot 202. The mechanical tracking arm 206′ may have a designated mechanical tracking computer (not shown) or the device computer 218 may be used to perform the calculations to determine the POSE of the digitizer probe 130′. The surgical system 200 may further include a bone motion monitor to monitor any movement of the bone relative to the fixation hardware, and may further include registration recovery markers for re-registering the bone in the event the bone moves beyond a specified threshold. In this embodiment, the bones may be first fixed relative to the surgical robot with fixation hardware (e.g., the bones are fixed to the base of the robot with pins, rods, and clamps). Next, the mechanical digitizer is used to designate to the entry and exit points for the tunnels to be created in the bone. The location of the entry and exit points are recorded and the vector therebetween is calculated in the device computer 218 or mechanically tracking computer. The surgical robot, using active or haptic control, then creates the tunnels along the vectors as described above. The ligament graft G is secured in the bone using fixation hardware 12 and the procedure is complete.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method for location placement of a first tunnel in a bone of a patient, said method comprising:
designating with a digitizer a start point for a first tunnel entry site for the first tunnel in the first bone;
designating with the digitizer an end point where the first tunnel terminates on the first bone; and
wherein the start point and the end point designate the location placement of the first tunnel.

2. The method of claim 1 further comprising supplying the start and end points to a robotic drill as a first vector to drill the first tunnel, and operating the robotic drill to drill the first tunnel.

3. The method of claim 1 further comprising:
designating with the digitizer a start point for a second tunnel entry site for a second tunnel in a second bone;
designating with the digitizer an end point where the second tunnel terminates on the second bone; and
wherein the start and end points designate the location placement of the second tunnel.

4. The method of claim 1 further comprising placing the ligament or tendon graft in the joint and securing a set of opposing ends of the graft with securements.

5. The method of claim 1 wherein the patient's first bone and second bone are a femur and tibia, respectively.

6. The method of claim 1 wherein the graft is an anterior cruciate ligament (ACL).

7. The method of claim 1 further comprising securing a tracking reference device to each of the first bone and a second bone that form the joint prior to designating the start point.

8. The method of claim 1 wherein the robotic drill is a surgical robot having an end-effector.

9. The method of claim 8 wherein the surgical robot automatically controls the end-effector along the first vector to drill the first tunnel.

10. The method of claim 8 further comprising:
automatically positioning the end-effector proximal to the first bone and aligned with the first vector; and
hand-guiding the end-effector into the first bone to create the tunnel while the surgical robot maintains the end-effector along the first vector.

11. A computer-assisted surgical system, comprising:
a tracking system to track the position of the digitizer of the method of claim 1;
a computing system having one or more computers with software, wherein said one or more computers record the location of the designated start point and the designated end point and calculates the first vector between the start point and the end point; and
wherein the computer supplies the vector to the robotic drill for drilling a tunnel.

12. The system of claim 11 wherein the tracking system comprises a tracking system computer, wherein the tracking system computer is one of the one or more computers of the computing system.

13. The system of 11 wherein the one or more computers records the location of the designated start point, end point, or vector relative to the coordinates of a tracking reference device attached to a bone.

14. The system of claim 13 wherein the tracking system tracks the location of the designated start point, designated end point, or calculated vector in real-time based on a position of the tracked reference device.

15. The system of claim 11 wherein the robotic drill is a surgical robot having an end-effector.

16. The system of claim 15 wherein the surgical robot provides active control to the end-effector to automatically create the tunnel along the first vector.

17. The system of claim 15 wherein the surgical robot provides semi-active control to the end-effector, wherein the surgical robot maintains the end-effector along the calculated vector while a user hand-guides the end-effector to create the tunnel.

18. The system of claim 11 wherein the tracking system is a mechanical tracking system having a plurality of links, joints, and encoders to track the digitizer, wherein the digitizer is attached to a distal end of the mechanical tracking system.

19. The method of claim 2 wherein the first vector provides a direction and length for the robotic drill.

20. The method of claim 3 further comprising supplying the start and end points to the robotic drill as a second vector to drill the second tunnel, and operating the robotic drill to drill the first tunnel.
